(19) United States
(12) Patent Application Publication
(10) Pub. No.: US 2015/0373923 A1
(43) Pub. Date: Dec. 31, 2015

(54) TREATED SPROUT PLANTS WITH DECREASED BACTERIAL VIABILITY AND METHODS AND APPARATUSES FOR MAKING THE SAME

(71) Applicant: EP Technologies LLC, Akron, OH (US)

(72) Inventors: James Ferrell, Stow, OH (US); Nicholas R. Lee, North Canton, OH (US); Daphne Pappas Antonakas, Hudson, OH (US); Sameer Kalghatgi, Copley, OH (US); Tsung-Chan Tsai, Cuyahoga Falls, OH (US); Robert L. Gray, Hudson, OH (US)

(21) Appl. No.: 14/753,969

(22) Filed: Jun. 29, 2015

Related U.S. Application Data
(60) Provisional application No. 62/018,179, filed on Jun. 27, 2014.

(51) Int. Cl. A01G 7/06; A01H 5/10; A01G 1/00

(52) U.S. Cl. CPC A01G 7/06; A01G 1/001; A01H 5/10

(57) ABSTRACT

Plasma treatment devices for reducing bacterial viability, enhancing stem growth, enhancing root growth or increasing germination times are disclosed herein. In addition, a treated sprout plant treated with a plasma activate medium or plasma with decreased bacterial viability relative to an untreated sprout plant. The treated sprout plant has at least a 1-log reduction in bacterial viability relative to the untreated sprout plant. Methods and apparatuses of producing the treated sprout plant are also provided.

### RELATED APPLICATIONS

This application claims priority to and the benefits of U.S. Provisional Application Ser. No. 62/018,179, filed Jun. 27, 2014, titled TREATED SPROUT PLANTS WITH DECREASED BACTERIAL VIABILITY AND METHODS AND APPARATUSES FOR MAKING THE SAME, which application is incorporated herein by reference in its entirety.

### FIELD OF THE DISCLOSURE

The disclosure relates to treated sprout plants. Particularly, the disclosure relates to treated sprout plants having decreased bacterial viability and methods and apparatuses for making the same.

### BACKGROUND

The Centers for Disease Control and Prevention estimate approximately 48 million Americans get sick, 128,000 are hospitalized, and 3,000 die from food-borne diseases in a year. According to the FDA, 131 produce-related outbreaks, attributed to both domestic and imported fresh produce between 1996 and 2010, resulted in 14,132 illnesses, 1,360 hospitalizations and 27 deaths. Food safety practices have advanced in recent years, but despite increased efforts, outbreaks caused by contamination of multiple types of sprout plants continue to occur. Current methods of sterilizing or decontaminating sprout plants include chemical treatment, heat treatment and irradiation.

Plasma is a weakly ionized gaseous medium that contains free electrons, ions and neutral particles. Non-thermal plasmas are generated when a gas is exposed to an electric field formed between two electrodes, one of which may be grounded. Upon the application of the electric field, the gas molecules and atoms generate free electrons, ions and radicals that participate in reactions within the gas phase and also with materials in contact with the plasma. A non-vacuum plasma system that operates at or below room temperature and at atmospheric pressure conditions is presented for consideration in this application.

### SUMMARY

Disclosed herein are treated sprout plants with decreased bacterial viability. Also disclosed are methods and apparatuses for producing the sprout plants.

Also disclosed are treated sprout plants with enhanced viability and methods and apparatuses for enhancing production of sprout plants.

In one aspect, a treated sprout plant with decreased bacterial viability is disclosed. The treated sprout plant has significantly reduced bacterial viability relative to an untreated sprout plant. The treated sprout plant has at least a 1-log reduction in bacterial viability relative to the untreated sprout plant.

In another aspect, a method of increasing root length of a sprout plant produced from a bean is provided. The method includes the step of exposing a bean of a sprout plant to plasma.

In still another aspect, a method of increasing stem length of a sprout plant is provided. The method includes the step of exposing a bean of a sprout plant to direct plasma.

In yet another aspect, a method of increasing germination of a bean of a sprout plant is provided. The method includes the step of exposing a bean of a sprout plant to direct plasma.

In still another aspect, a method of increasing water retention in a bean of a sprout plant is provided. The method includes the step of exposing a bean of a sprout plant to direct plasma.

In another aspect, an apparatus for decontaminating a sprout plant is provided. The apparatus includes a plasma source, a medium generator, and a plasma activated medium applicator. The plasma activated medium applicator is adapted to apply the plasma activated medium to a sprout plant.

Plasma treatment devices for reducing bacterial viability, enhancing stem growth, enhancing root growth or increasing germination times are disclosed herein. An exemplary embodiment includes a cylindrical housing that rotates about an axis and one or more medium activation nozzles. The medium activation nozzles include a housing, a medium inlet, an outlet, a high voltage electrode and a medium passage located at least partially between the housing and the high voltage electrode and extending from the medium inlet to the medium outlet. When the high voltage electrode is energized, a non-thermal plasma is generated in at least a portion of the medium passage. At least a portion of the medium is activated by passing through the non-thermal plasma and creates reactive species in the activated medium.

Another exemplary embodiment includes a contact surface for moving crops or seeds and a plurality of medium activation nozzles. The plurality of medium activation nozzles include a housing, a medium inlet, an outlet, a high voltage electrode, and a medium passage located at least partially between the housing and the high voltage electrode. When the high voltage electrode is energized, a non-thermal plasma is generated in at least a portion of the medium passage; and at least a portion of the medium is ionized to create reactive species in the medium.

Another exemplary embodiment includes a cylindrical drum having a first portion and a second portion. The first portion separates from the second portion to allow access to an inner portion of the cylindrical drum. A cylindrical drum drive for rotating the cylindrical drum is also provided. the exemplary embodiment includes one or more medium activation nozzles located at least partially within the cylindrical drum. The medium activation nozzles includes a housing, an electrode, a fluid inlet, a fluid outlet and a medium passage extending from the fluid inlet to the fluid outlet. At least a portion of the medium passage is adjacent to the high voltage electrode. Energizing the high voltage electrode produces plasma in at least a portion of the medium passage and the medium flows through at least a portion of the plasma. At least a portion of the medium is ionized to activate the medium.

Further areas of applicability of the present disclosure will become apparent from the detailed description, drawings, and claims provided hereinafter. It should be understood that the detailed description, including disclosed embodiments and drawings, are merely exemplary in nature, are only intended for purposes of illustration and are not intended to limit the scope of the invention, its application or use.

### BRIEF DESCRIPTION OF THE DRAWINGS

FIG. 1 depicts an exemplary embodiment of a direct plasma source.

FIG. 2 depicts an exemplary embodiment of an indirect plasma source.

FIG. 3 shows the average log reduction for E. coli on mung beans following dielectric barrier discharge (DBD) exposure using ambient air as the operating gas.

FIG. 4 shows the average log reduction for E. coli on mung beans following non-thermal plasma-activated water mist exposure.

FIG. 5 shows the mung bean germination percentages following DBD plasma exposure with varied gas mixtures.

FIG. 6 shows the mung bean plant stem and root growth after DBD plasma exposure of mung beans with varied gas mixtures.

FIG. 7 shows the water retention in mung beans exposed to DBD plasma with various gas mixtures.

FIG. 8 shows an example of an exemplary embodiment of an apparatus for treating food products which includes a conveyor belt.

FIGS. 9-11 show another example of an exemplary embodiment of an apparatus for treating food products which includes a tumbler.

FIG. 10A illustrates yet another example of an exemplary embodiment of an apparatus for treating food products which includes a tumbler.

FIG. 12 shows the mung bean plant stem and root growth after DBD plasma exposure of mung beans with varied gas mixtures treated in the tumbler of FIGS. 9-11.

FIG. 13 shows the mung bean germination percentages following DBD plasma exposure with varied gas mixtures treated in the tumbler of FIGS. 9-11.

### DETAILED DESCRIPTION

Disclosed herein are treated sprout plants with significantly decreased bacterial viability relative to untreated sprout plants.

“Treated sprout plants” are those sprout plants which are subjected to a treatment due to the actions of a human being, i.e., where a human has taken an action to direct treatment to the particular sprout plant. An “untreated sprout plant” is the same sprout plant which has not been subjected to the particular human-directed treatment, or another human-directed treatment which is intended to produce a similar effect on the sprout plant.

In particular embodiments, “treated sprout plants” are those which have been exposed either directly to non-thermal plasma (e.g., DBD jet or planar DBD electrode) or to a plasma activated medium (e.g., water mist) and “untreated sprout plants” are those which have neither been directly exposed to non-thermal plasma nor exposed to a plasma activated medium.

Applicants have discovered that non-thermal plasma can act as a disinfectant that can kill oral and gastrointestinal tract pathogens found on the surfaces of sprout plants. When non-thermal plasma is exposed to air as an initial gaseous medium, reactive oxygen and nitrogen species are produced. These species are the driving force behind antimicrobial activity within non-thermal plasmas.

In some embodiments, sprout plants are exposed to a direct plasma source, e.g., DBD jet or planar DBD electrode. Direct plasma sources may include, without limitation, coronas, jets and surface DBDs. Direct plasma sources can be used under any equipment settings that decrease bacterial viability on a sprout plant without producing significant tissue damage. In some embodiments, direct plasma source settings include a voltage that ranges from 3 kV-30 kV, a frequency that ranges from 50 Hz-10 kHz, a pulse duration that ranges from 1 ns-1 ms, and a duty cycle that ranges from 1%-100%. Any gas that is safe and effective for plasma activation may be used to ignite the plasma. In some embodiments, the gas is one or more of air, helium, argon, oxygen, nitrogen, water vapor, vaporized ethanol and mixtures and combinations thereof. Direct plasma treatment is appropriate for any crop tissue that is not highly susceptible to damage (e.g., beans of sprout plants and sprout plants).

In other embodiments, sprout plants are exposed to a plasma activated medium. Plasma activated medium may be either activated by exposure to direct plasma or exposure to afterglow. Medium activated by exposure to direct plasma are those which come into direct contact with a plasma whereas medium activated by exposure to after-glow is done by passing plasma through a filter and contacting the medium with the species that passed through the filter and which is referred to as medium subjected to indirect plasma treatment. Exemplary embodiments for direct plasma and for afterglow are shown and described in U.S. patent application Ser. No. 13/843,189, titled Methods and Solutions for Killing or Deactivating Spores, which is incorporated by reference herein in its entirety.

Medium includes both liquids and/or fluids and gases. In particular embodiments, gaseous medium includes one or more of air, helium, argon, neon, xenon, oxygen, nitrogen, vaporized water, ethanol and other vaporized liquids, and mixtures thereof. In some embodiments, the liquid and/or fluid medium is one or more of water, saline, ethanol and other organic solvents, water-based or non-aqueous solutions containing salts or acids, aerosolized liquids or mixtures thereof dispersed in the above mentioned gaseous medium.

Sprout plants with “decreased bacterial viability” are those sprout plants with fewer bacteria present following treatment than prior to treatment. Sprout plants are particularly susceptible to bacterial growth given the environment in which sprout plants are grown. The high susceptibility of sprout plants is heightened even further for sprout plants grown organically, i.e., without treatment with inorganic chemicals. Thus, sprout plants with decreased bacterial viability, especially sprout plants grown organically, are desirable. Decreased bacterial viability can be measured by means known in the art. For example, tissue from both treated and untreated sprout plants can be removed, solubilized, and bacterial growth of the solution measured, e.g., on an agar plate, can be quantified. Quantification can be done by determining the number of colony forming units (CFUs) present from each of the samples taken. A logarithmic (log) ratio can then be calculated as a measure of the difference between bacterial viability of treated and untreated samples. In particular embodiments, treated samples have at least a 1-log difference, including at least a 2-log difference, including at least a 3-log difference, including at least a 4-log difference, including at least a 5-log difference, including between a 5-log and a 6-log difference, including about a 5.5 log difference, also including at least a 6-log difference, including at least a 7-log difference, including at least an 8-log difference in bacterial viability relative to untreated samples. In the food industry, a 5-log or greater decrease in bacterial viability is considered sufficient to provide for decontamination of a sprout plant. Thus, in some embodiments, sprout plants disclosed herein are “decontaminated” through exposure to a plasma treatment disclosed herein.

Bacteria with decreased viability as a result of plasma treatment of sprout plants include any food-borne bacterium, ranging from spores to vegetative cells to biofilms, which can reside on a sprout plant. In particular embodiments, the bacteria with decreased viability include one or more of E. coli, S. aureus, Listeria and Salmonella.

In some embodiments, in addition to or separately from significantly decreased bacterial viability, treated sprout plants have improved phenotypic characteristics. Such characteristics include, but are not limited to, one or more of increased root length, increased stem length, increased germination rate and increased water retention. Improvements in the phenotypic characteristics can decrease the time required to grow a sprout plant to a particular state, produce larger sprout plants over a given time, or both. Given that sprout plants have relatively short growing periods compared to other plants, improvements in phenotypic characteristics can produce results that are quickly measurable and significantly impact the quality of a sprout plant, the time required for growth, or both. The characteristics may be improved in treated sprout plants by or to absolute amounts (e.g., a particular number of centimeters or to a particular percentage of germination), or, in some embodiments, by a relative amount (e.g., a particular percentage). For example, in some embodiments, treated sprout plant beans will produce sprout plants with an increase in root length of at least about 10%, including at least about 15%, including at least about 20%, including at least about 30%, and including at least about 40% relative to the root length of an untreated sprout plant. In some embodiments, treated sprout plant beans will produce sprout plants with increased stem length in addition to or separately from the increased root length. The stem length may be increased by at least about 5%, including at least about 10%, including at least about 15%, and including at least about 20% relative to the stem length of an untreated sprout plant. In some embodiments, treated sprout plant beans have increased germination rates in addition to or separately from the increased root and/or stem lengths of the sprout plants produced from treated sprout plant beans. In some embodiments, the germination rate of treated sprout plant beans is at least about 78%, including at least about 79%, including at least about 80%, including at least about 81%, including at least about 82%, including at least about 83%, including at least about 84%, including at least about 85%, including at least about 86%, including at least about 87%, including at least about 88%, including at least about 89%, including at least about 90% within 24 hours of exposure to treatment. In some embodiments, treated sprout plant beans have increased water retention in addition to or separately from the increased root lengths, stem lengths and/or germination rates of the treated spout plant beans or the plants produced there from. In some embodiments, the water retention of treated sprout plant beans is increased by at least about 5%, including at least about 10%, including at least about 15%, including at least about 20%, including at least about 25%, including at least about 35%, and including at least about 40% relative to untreated sprout plant beans.

The treated sprout plants disclosed herein may be produced by applying a method which, in one aspect, includes the step of exposing a sprout plant to a plasma activated medium. In another aspect, the method includes the step of exposing a sprout plant to a plasma. The method can be used to produce treated sprout plant beans and corresponding sprout plants with one or more of significantly decreased bacterial viability, and improved phenotypic characteristics including increased root length, increased stem length, increased germination, and increased water retention. The plasma activated medium may be any medium that is activated by exposure to a plasma. In some embodiments, the plasma activated medium is a gas or a fluid. Any gas or fluid that can be activated by exposure to a plasma and to which sprout plants can safely be exposed can be used. In particular embodiments, the plasma activated medium is water. In particular embodiments, the plasma activated medium is a water mist.

Sprout plants with significantly decreased bacterial viability and/or one or more of the enhanced phenotypic characteristics described above are preferably produced by applying the method for a limited time period. In some embodiments, exposure is carried out for time periods of less than about a three minutes, including time periods of less than about one minute, including time periods of less than about 45 seconds, including time periods of less than about 30 seconds, including time periods of less than about 15 seconds. Exposure of sprout plants for these limited time periods can be used to produce sprout plants with greater than about a 1-log, including greater than about a 3-log, including greater than about a 5-log reduction in bacterial viability. In a particularly specific embodiment, a 5.5 log reduction in bacterial viability is produced in about 1 minute of exposure. The unexpectedly large reductions in bacterial viability over short exposure times allows for both fast processing of sprout plants and can aid in limiting the damage to sprout plants.

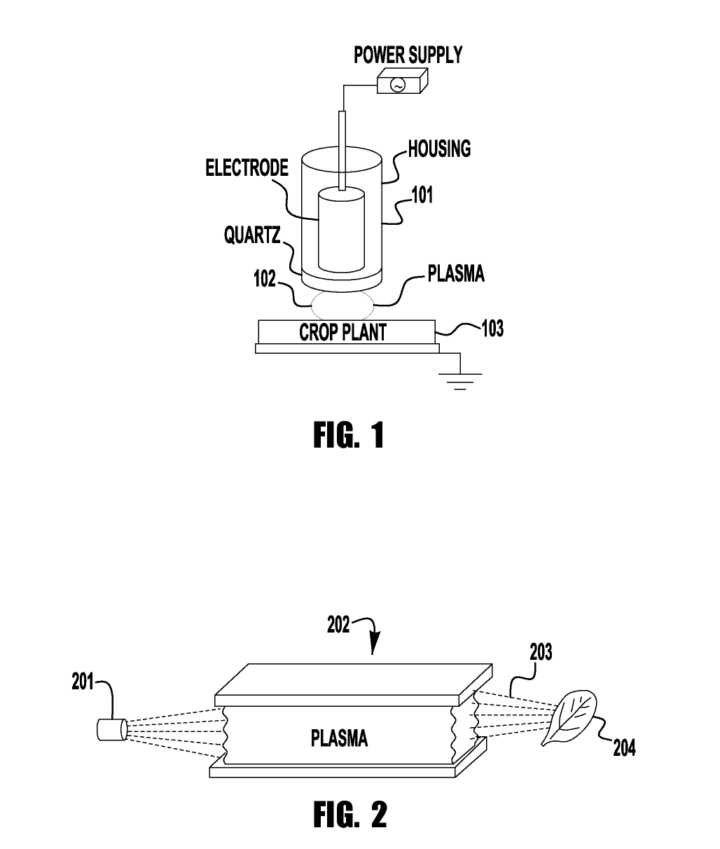

The methods may be carried out and/or the sprout plants produced by using an apparatus as disclosed herein. In some embodiments, an apparatus used to carry out the methods and/or for producing the sprout plants disclosed herein includes a plasma source, a medium generator, and a plasma activated medium applicator. In some embodiments, the plasma activated medium applicator is preferably adapted to apply the plasma activated medium to a sprout plant. Exemplary apparatuses for direct and indirect plasma treatments of sprout plants are shown in FIGS. 1-2, respectively. Referring to FIG. 1, an exemplary apparatus for direct plasma treatment is provided. The apparatus contains a plasma generator 101. The plasma generator 101 generates plasma in an air medium containing nitrogen and oxygen 102. The plasma is directed to a sprout plant 103. Plasma generator 101 is a dielectric barrier discharge (“DBD”) plasma generator. In some embodiments, the plasma generator is a plasma jet. In other embodiments, the plasma generator generates a corona discharge while in some other embodiments the plasma generator generates a plasma afterglow medium. In some embodiments, a filter (not shown) is included to generate indirect plasma which is applied directed to the sprout plant.

Referring to FIG. 2, another exemplary apparatus for plasma treatment is provided. The apparatus contains a medium generator 201. The medium generator 201 generates a medium, such as, for example, a mist of water droplets in air, which is passed through plasma generated by plasma generator 202. The medium is activated by plasma from the plasma source 202. The activated medium 203 is directed to a sprout plant 204. The sprout plant 204 is placed at a distance from the apparatus to allow efficient exposure of the sprout plant 204 to the activated medium 203, with limited to no damage to the sprout plant 204. Any appropriate distance may be used. In some embodiments, the sprout plant 204 is placed at a distance between about 2 mm and about 30 mm, including between about 5 mm and about 27 mm, including between about 10 mm and about 20 mm, including at about 15 mm, from the apparatus. Although specific ranges are disclosed herein, these distances may be increased by varying plasma settings and/or including one or more stabilizers in the medium that extend the life of the activated species. Accordingly, the distances are not limiting on the inventive concepts disclosed herein. The distance can be varied separately or in combination with varying a scale setting on the apparatus regulating the generation of the medium such that the activated medium 203 flows to the sprout plant 204 at an appropriate rate. In some embodiments, variations in the distance and the scale setting are carried out to produce a flow rate of the activated medium 203 to the sprout plant 204 of about 1 mg of the activated medium 203 per minute to about 20 mg of the activated medium 203 per minute, including about 2 mg to about 8 mg of the activated medium 203 per minute, including about 4 mg to about 6 mg of the activated medium 203 per minute, including about 5 mg of the activated medium 203 per minute.

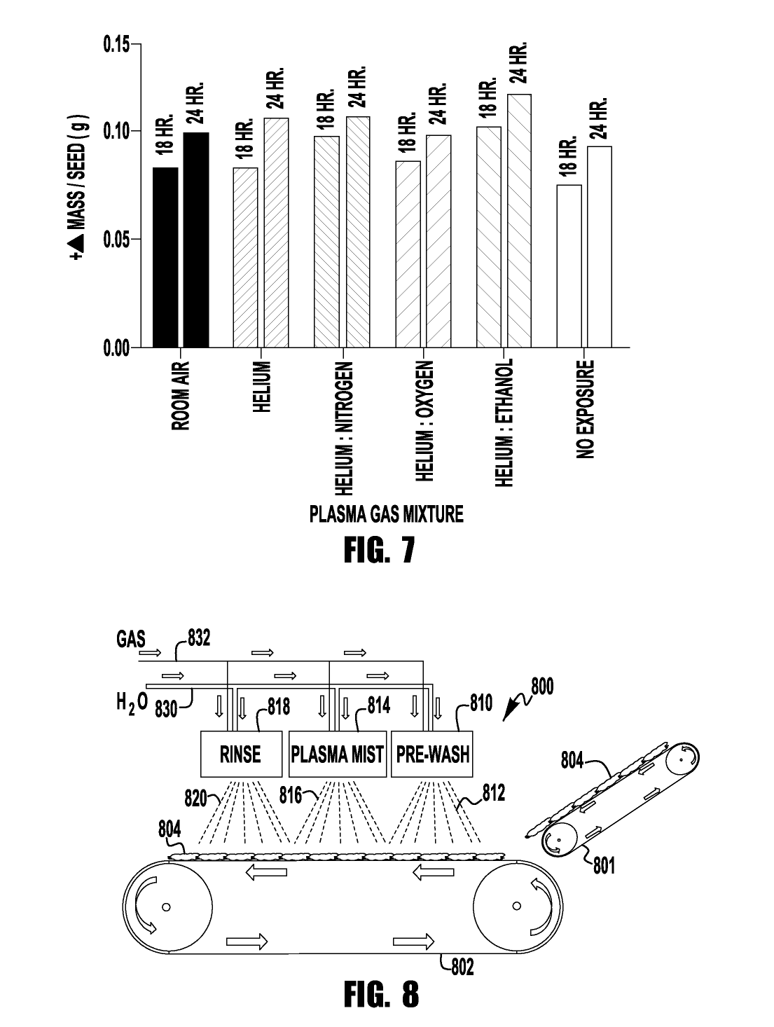

FIG. 8 illustrates and exemplary embodiment of a treatment apparatus 800 for treating crops, such as sprout plants that includes a conveyor system 801, 802. Treatment apparatus 800 includes a feed conveyor 801 that feeds a crop plant 804 to treatment conveyor 802 which moves the crop plants in direction F. The exemplary embodiment includes one or more pre-wash stations 810 that spray the crop 804 with a pre-wash to wash of dirt and contaminants. In some exemplary embodiments, conveyor 802 vibrates and flips the crop plant 804 around. Treatment apparatus 800 includes one or more plasma treatment stations 814. The exemplary plasma treatment station 814 provides a plasma activated medium in the form of a mist to the crop 804. In some embodiments, plasma treatment station 814 is a dry plasma treatment station and the plasma activated medium is a gas to the crop 814. In some embodiments, the plasma activated medium is activated by direct plasma and in some embodiments; the plasma activated medium is activated by indirect plasma. At rinse station 818, the crop 804 is rinsed, with for example, a water spray 820. In many cases it is not necessary to rinse the crop 804 after the crop 804 is treated with the plasma activated medium. Accordingly, in some embodiments, rinse station 818 is not used or required. In some embodiments, the crop 804 is completely rinsed prior to entering the plasma treatment station. In the exemplary embodiment, a water supply 830 and a gas supply 832 is provided to all of the stations. In some embodiments, the gas 832 supply is only supplied to the plasma treatment station 814. In some embodiments, the gas 832 may be any of the gases identified herein.

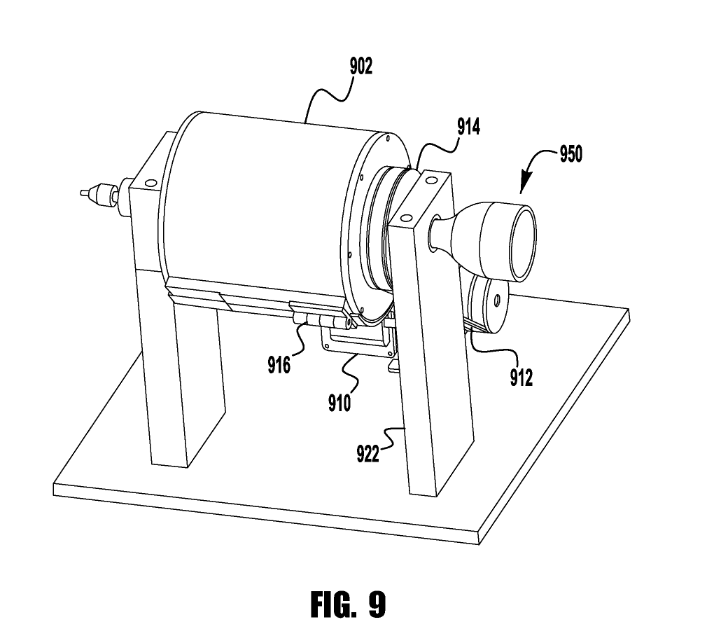

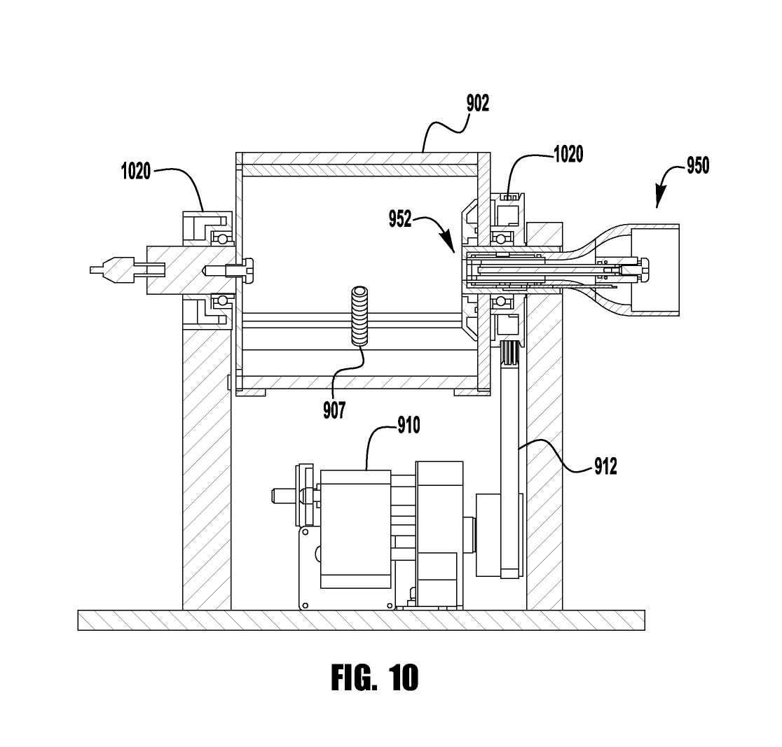

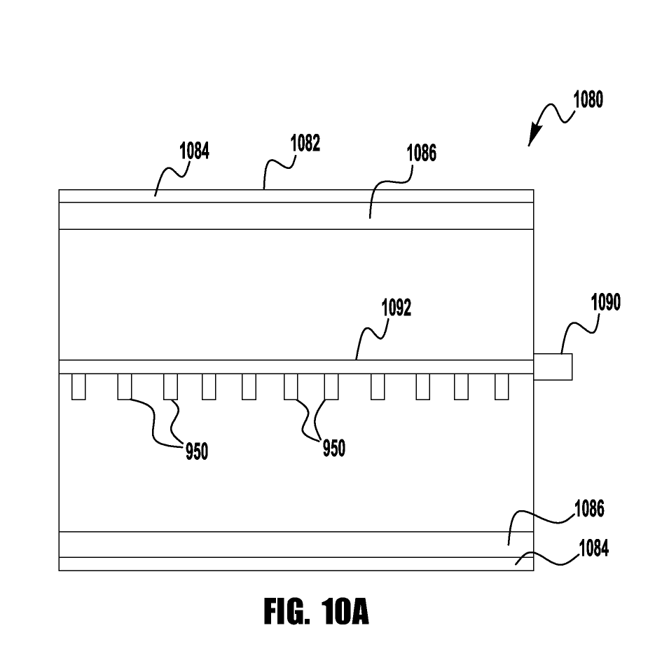

FIGS. 9, 10 and 11 illustrate an exemplary embodiment of a plasma treatment tumbler 900. Plasma treatment tumbler 900 includes a rotating drum 902. Rotating drum 902 may include screens and/or tumbling bars (not shown) on the interior to cause crops/seeds (not shown) to tumble, rotate, etc. to allow activated medium to contact all surfaces of the crop/seeds. Use of the term crop herein may include seeds. Rotating drum 902 includes hinges 916 and a latch 907 (FIG. 10) so that rotating drum 902 may be opened to add the crops and closed and locked so the rotating drum 902 may be rotated without spilling the crop. Plasma treatment tumbler 900 includes a pair of support legs 922 and bearings 1020 that allow rotating drum 902 to rotate. A motor 910 turns a belt 912 to rotate drum 902. In addition, plasma treatment tumbler 900 includes a medium activation nozzle 950.

FIG. 10A illustrates another exemplary embodiment of a plasma treatment tumbler 1080. The exemplary plasma treatment tumbler 1080 is similar to the exemplary plasma treatment tumbler 900, however, only the rotating drum portion 1082 is illustrated. Plasma treatment tumbler 1080 has a rotating drum 1082 and includes a plurality of medium activation nozzles 950, which allows a larger volume of crops/seeds to be treated at a time. In addition, in some embodiments, the activated medium may be more evenly dispersed with multiple medium activation nozzles 950. Plasma treatment tumbler 1080 also includes an optional screen 1084 and optional tumbling bars 1086. One or more connection ports 1090 allow for the connection of the plasma treatment tumbler to a medium source and connection to a high voltage source.

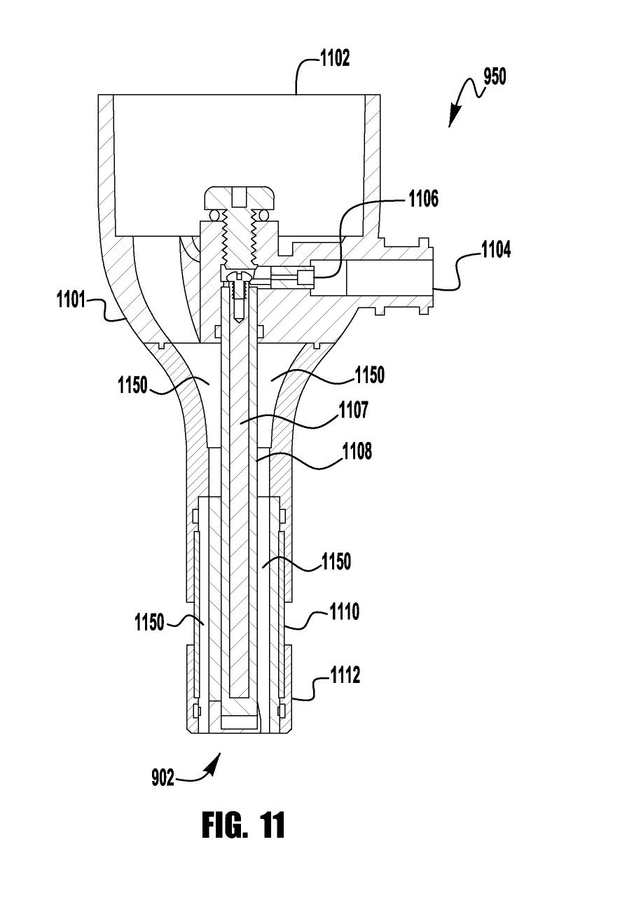

FIG. 11 illustrates an exemplary embodiment of a medium activation nozzle 950. Nozzle 950 includes a housing 1101 and medium inlet port 1102. In the exemplary nozzle 950, medium inlet port 1102 is a mist inlet port, however, in some embodiments, medium inlet port 1102 may be a gas inlet port, a liquid inlet port, a vapor inlet port or the like. Nozzle 950 includes a high voltage connection port 1104 to allow a high voltage connector (not shown) to place a high voltage source (not shown) in circuit communication with conductor 1106. Conductor 1106 is in circuit communication with electrode 1107. A dielectric material 1108 surrounds electrode 1107. A ground dielectric 1110 and spacer cap 1112 are also included. A medium passage 1150 is located within the housing 1101 and provides a path for the medium to be activated to pass from the medium inlet port 1102 to outlet 902. The medium passage 1150 extends between housing 101 and the outside of dielectric 1108.

During operation the crop/seeds to be treated is loaded into rotating drum 902 which starts to rotate. A medium, such as mist, to be activated flows into medium inlet 1102. Electrode 1107 is energized to produce plasma. Electrode 1107 may be powered as described above and in the references included by reference herein to generate plasma within medium passage 1150. As the medium passes through the plasma, it is activated. The activated medium flows into rotating drum 902 to treat the crops/seeds that are tumbling around within the rotating drum 902. After a set period of time, the power source is turned off, the drum 902 stops rotating and the crop/seeds may be removed.

Surprisingly, it has been discovered that one treatment with plasma activated medium or contacted with plasma can reduce bacterial viability, increase water retention, increase germination, and increase stem and root growth. Each of which is highly beneficial on its own and in combination is extremely beneficial as the crops/seeds can be grown significantly faster with less bacterial viability resulting in greater crop yields.

It should be understood that the apparatus need not be in any particular shape or size. The apparatus need only contain elements that allow for activation of a medium by plasma and the exposure of a sprout plant to the activated medium. In that regard, the apparatus may be a single or multiple units, and can contain either or both direct and indirect plasma treatment options. In some embodiments, the apparatus is designed for use by employees at food processing facilities. In some of these embodiments, the apparatus is designed as a glove that, in some embodiments, fits over the hand and which can direct plasma treatment to sprout plants handled by the food worker. In other embodiments, such as shown in FIG. 8, the apparatus is a part of a conveyor system that allows for the treatment of sprout plants that pass on the conveyor system. The apparatus may be placed in any orientation relative to the sprout plants that pass on the conveyor system. In a particular embodiment, the apparatus is located above a conveyor belt such that sprout plants are treated as they pass under the plasma activated medium. While apparatuses for indoor use have generally been described herein, the apparatus can be used indoors or outdoors. In that regard, apparatuses designed for outdoor use can be of any form that contains a power source sufficient to power the apparatus whereby a non-thermal plasma is generated. The power source could be integrated into the apparatus or provided on a use basis. In some embodiments, the power source is selected from microsecond, sinusoidal, nanosecond, and radiofrequency (RF) power sources. In some embodiments, the apparatus is designed for use on sprout plants in the field. In some embodiments, the apparatus treats beans before planting the beans in the field, and, in some embodiments, the planter includes an apparatus, which treats the beans just prior to planting.

### EXAMPLES

The following examples illustrate specific and exemplary embodiments, features, or both, of the methods and apparatuses disclosed herein. The examples are provided solely for the purpose of illustration and should not be construed as limitations on the present disclosure.

### Example 1

### Plasma Treatment Decreases E. coli Viability on Mung Beans

A. Direct Plasma Treatment Decreases E. coli Viability on Mung Beans.

This Example shows the effects of direct plasma treatment on the viability of E. coli present on mung beans.

Inoculation of mung beans. Escherichia coli (ATCC 35150) cultures were grown to stationary phase in tryptic soy broth (TSB; Difco, Becton Dickinson, Franklin Lakes, N.J.). Aliquots of the prepared cultures were mixed into a volume of sterile Milli-Q water (Millipore, Billerica, Spain) that yields an overall bacterial inoculum percentage of 2%. A large number of mung beans (500-600) were soaked in the prepared diluted cultures for 25 minutes, drained and dried in a laminar flow hood overnight.

Non-thermal plasma treatments. Non-thermal, DBD plasma was generated from three main gas mixtures: helium-nitrogen, helium-oxygen and helium-ethanol vapor using EP Technologies' plasma system, a schematic of which is shown in FIG. 1. The total electrode length and diameter was 5 cm and 2.54 cm, respectively, and was connected to a high voltage power supply (1-20) kV with adjustable parameters. To promote the formation of nitrogen and oxygen antimicrobial species (RONS), varied percentages (0.1%-20%) of N₂ or O₂ gas were added to the gas mixture. These gas mixtures may be adjusted as needed to yield optimum concentrations for decontamination of sprout plants.

Mung beans were placed in a Teflon® holder at a distance of 2 mm from the upper metal electrode and were exposed to non-thermal plasma s at intervals that ranged from 10 seconds to 2 minutes in batches of 20 beans per exposure duration group. Temperature measurements were taken throughout the experiments to ensure that the germicidal characteristics observed in exposed beans was not biased by elevated temperatures. Exposed beans were placed into centrifuge tubes and supplied with sterile de-ionized water. The tubes were then placed in an incubator and incubated at 37° C. until extraction.

Detection of surviving pathogenic bacteria. Bacterial growth and viability was assessed on days 1, 2, and 5 post-plasma exposure. Experimental beans were removed from the incubator and gently stomached in a sterile 0.1% peptone solution for 2 minutes. The homogenate was serially diluted in the peptone solution and spread-plated onto tryptic soy agar (TSA). The plates were then incubated at 37° C. for 24 hours. Bacterial cell enumeration was collected the following day using a plate reader and computational analysis programming. Viable organisms were spot checked as a back-up method for data collection. Data was analyzed using GraphPad Prism and InStat graphical and statistical software (GraphPad Software, Inc., La Jolla, Calif.).

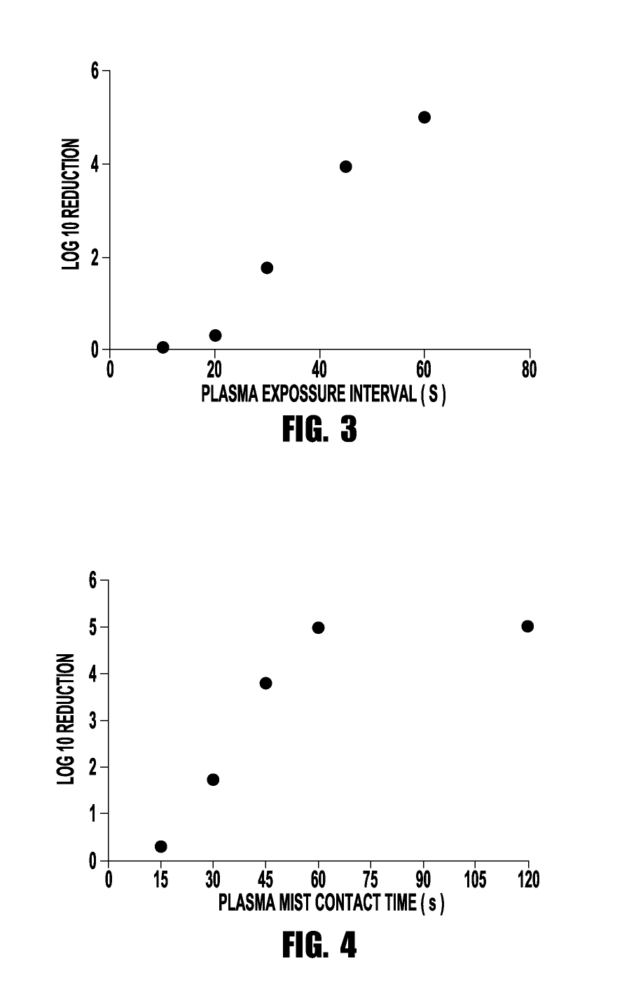

Results of the experiments are shown in FIG. 3. As shown in FIG. 3, mung beans exposed to direct plasma treatment derived from a gas mixture of 90% helium and 10% nitrogen for 1-minute exhibited an approximate 6-log reduction in E. coli viability.

B. Indirect Plasma Treatment Decreases E. coli Viability on Mung Beans.

This Example shows the effects of plasma mist treatment on the viability of E. coli present on mung beans.

Mung beans were inoculated with E. coli as described in the previous Example. To generate a plasma mist, an Ultrasonic 360 humidifier (Safety 1st; Columbus, Ind.) was connected to plastic tubing and fed into a custom small-scale plasma reactor (a schematic view is shown in FIG. 2). The plasma generator configuration consisted of two parallel brass plate electrodes that have an area of 40 mm×45 mm and thickness of 5 mm. Polyetherimide was used as the housing material of the electrodes. The upper electrode was connected to a high voltage power supply (1-20 kV) with operating parameters that can be adjusted while the lower electrode is grounded. The adjustable outlet of the setup released plasma-activated water mist onto inoculated beans fixed 2 mm beneath the opening. Plasma-activated water mist was applied to the inoculated mung beans at intervals from 15 seconds to 2 minutes. Treated beans were tested for E. coli viability as described in the previous Example.

Results of the experiment are shown in FIG. 4. As shown in FIG. 4, mung beans exposed to indirect plasma treatment for about 1-minute exhibited about a 5-log reduction in E. coli viability.

### Example 2

### Plasma Treatment Enhances Mung Bean Phenotypic Characteristics

### A. Plasma Treatment Enhances Mung Bean Germination.

This Example shows the effects of plasma treatment on mung bean germination. The power source was set at scale 15, 19.1 kHz frequency and ˜80% duty cycle.

At 18 hours and 24 hours post direct plasma exposure, exposed mung beans were evaluated for germination. Germination was identified as extension or eruption of the radical through the bean coat by more than 3 mm.

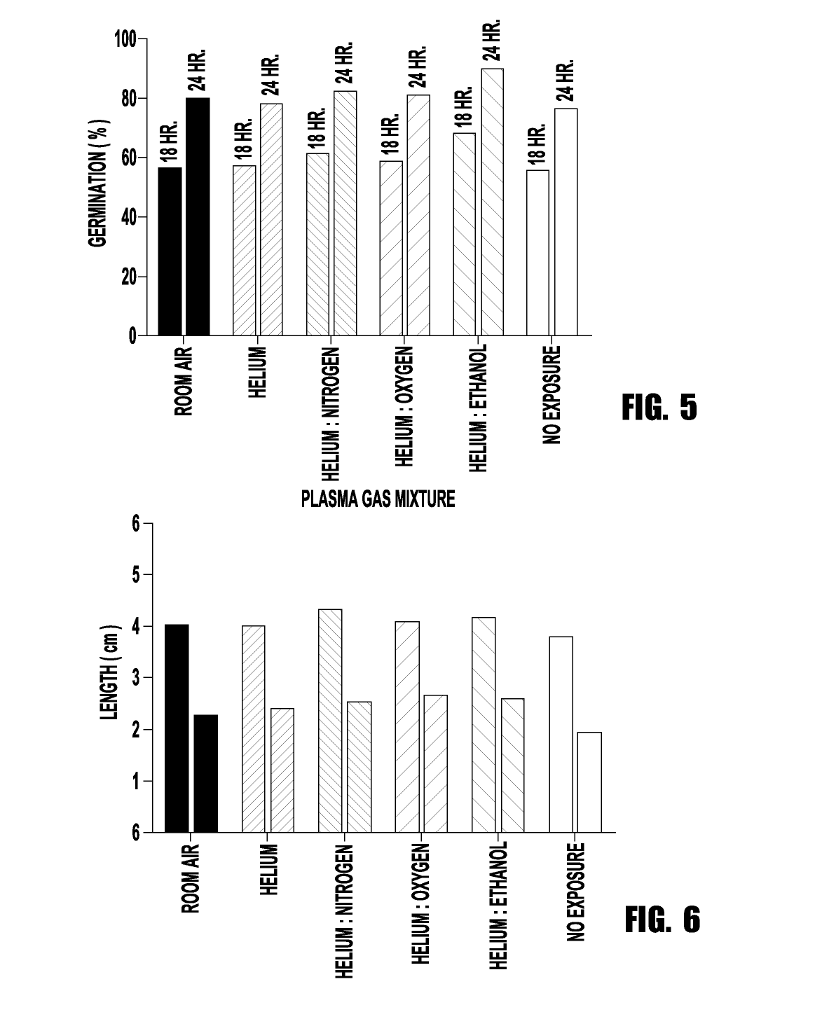

Results of the experiments are shown in FIG. 5. As shown in FIG. 5, mung beans exposed to plasma treatment using various gases all demonstrated increased germination relative to unexposed mung beans (No Exposure) at both 18 (18 hr) and 24 hours (24 hr) after direct plasma exposure. At 24 hours, germination ranged from about 78% (Helium) to about 89% (Helium:Ethanol vapor) versus about 76% for untreated beans (No Exposure).

### B. Plasma Treatment Enhances Mung Bean Stem and Root Growth.

This Example shows the effects of plasma treatment on mung bean plant stem and root growth. The power source was set at scale 15, 19.1 kHz frequency and ˜80% duty cycle.

At 5 days after plasma exposure, the sprouts (stems) and roots of plants produced from the germinated mung beans were measured. Measurements were obtained in 20 bean cohorts.

Results of the experiments are shown in FIG. 6. As shown in FIG. 6, mung bean plants exposed to plasma treatment using various gases all demonstrated increased stem (Stem) and root (Root) growth relative to unexposed mung bean plants (No Exposure). The increase in stem (Stem) growth was about 6% (Room Air) to about 14% (Helium:Nitrogen). The increase in root (Root) growth was about 18% (Room Air) to about 37% (Helium:Oxygen).

### C. Plasma Treatment Enhances Mung Bean Water Retention.

This Example shows the effects of plasma treatment on mung bean water retention. The power source was set at scale 15, 19.1 kHz frequency and ˜80% duty cycle.

Water retention measurements were carried out at 18 and 24 hours after plasma treatment of mung beans. Water retention was defined as the change in mass of the bean composite divided by the number of beans. A high precision balance (Mettler Toledo, model PL303) was used to weigh the beans before and after water retention. The beans were drained thoroughly and allowed to air dry prior to measurement. Measurements were obtained in 20 bean cohorts. Afterwards, all remaining water was pooled and subtracted from the composite value for each experimental bean cohort average. The beans were watered on a consistent daily schedule and maintained in Sprout Master™ sprouting trays (Sprout People, Madison, Wis.) over the course of the experiment.

Results of the experiments are shown in FIG. 7. As shown in FIG. 7, mung beans exposed to plasma treatment using various gases all demonstrated increased water retention at 18 (18 hr) and 24 hours (24 hr) after plasma exposure relative to unexposed mung beans (No Exposure). At 24 hours, the increase in water retention was about 5% (Room Air) to about 26% (Helium:Ethanol vapor).

### D. Plasma Tumbler Treatment Enhances Mung Bean Stem and Root Growth.

This Example shows the effects of plasma tumbler treatment on mung bean plant stem and root growth. The apparatus was configured in accordance with the tumbler described above. The power source was an AC sinusoidal power source set at a scale 15, 19.1 kHx, 80% DC, 25 W for all of the experiments. Mist was passed through the nozzle and activated. Experiments were conducted exposing the mung bean sprouts to the activated mist for 30 seconds, 1 minute, 2 minutes and 3 minutes. A control with no exposure time was also conducted.

At 5 days after plasma exposure, the sprouts (stems) and roots of plants produced from the germinated mung beans were measured. Measurements were obtained in 30 bean cohorts for each range.

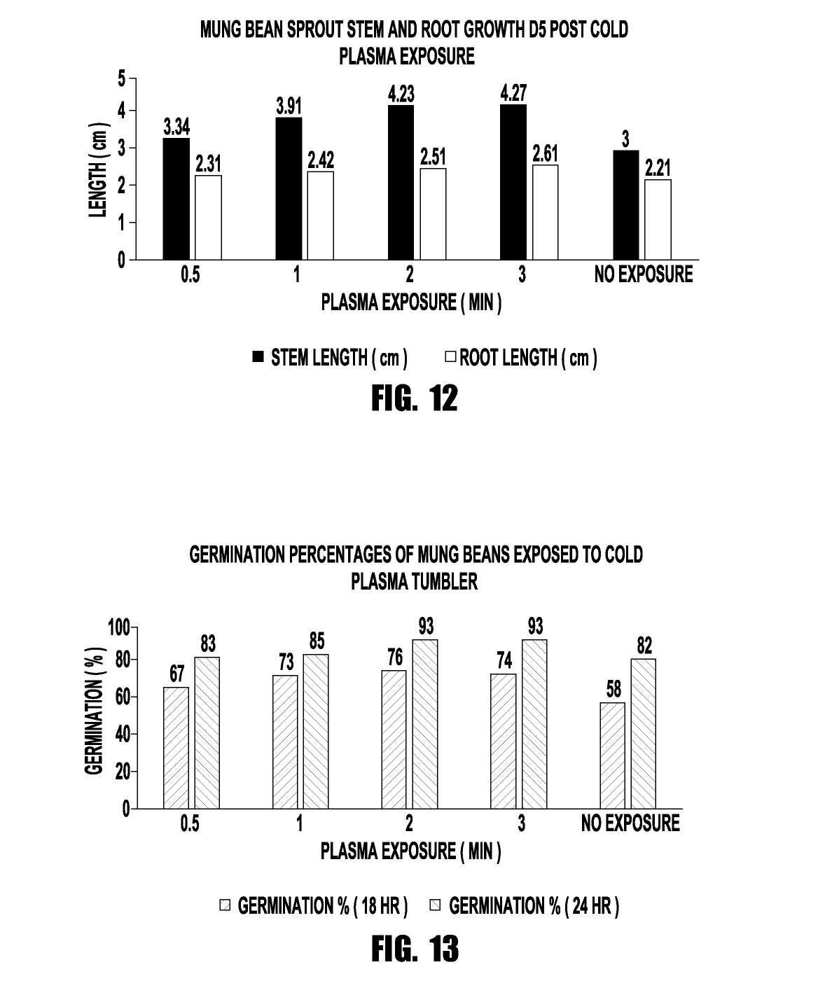

Results of the experiments are shown in FIG. 12. As shown in FIG. 12, mung bean plants exposed to plasma tumbler treatment using normal air all demonstrated increased stem (Stem) and root (Root) growth relative to unexposed mung bean plants (No Exposure). As shown in Table I, below, for plasma exposure time of 30 seconds, the increase in stem (Stem) growth was about 0.34 cm and the increase in root (Root) growth was 0.1 cm. For plasma exposure time of 1 minute, the increase in stem (Stem) growth was about 0.91 cm and the increase in root (Root) growth was 0.21 cm. For plasma exposure time of 2 minutes, the increase in stem (Stem) growth was about 1.23 cm and the increase in root (Root) growth was 0.3 cm, and for plasma exposure time of 3 minutes, the increase in stem (Stem) growth was about 1.27 cm and the increase in root (Root) growth was 0.4 cm.

TABLE I

| Plasma Exposure (Min) | +Stem Length (cm) | +Root Length (cm) |
|---|---|---|
| 0.5 | 0.34 | 0.1 |
| 1 | 0.91 | 0.21 |
| 2 | 1.23 | 0.3 |
| 3 | 1.27 | 0.4 |

### E. Plasma Tumbler Treatment Enhances Mung Bean Germination.

This Example shows the effects of plasma tumbler treatment on mung bean germination.

The power source was an AC sinusoidal power source set at a scale 15, 19.1 kHz, 80% DC, 25 W for all of the experiments. Mist was passed through the nozzle and activated. Experiments were conducted exposing the mung bean sprouts to the activated mist for 30 seconds, 1 minute, 2 minutes and 3 minutes. A control with no exposure time was also conducted.

At 18 hours and 24 hours post direct plasma exposure, exposed mung beans were evaluated for germination. Germination was identified as extension or eruption of the radical through the bean coat by more than 3 mm.

Results of the experiments are shown in FIG. 13. As shown in FIG. 13, mung beans exposed to plasma treatment using various gases all demonstrated increased germination relative to unexposed mung beans (No Exposure) at both 18 (18 hr) and 24 hours (24 hr) after direct plasma exposure as shown in Table II below, for a plasma exposure time of 30 seconds, the increase in germination at 18 hours was 9% and at 24 hours was 1%. For a plasma exposure time of 1 minute, the increase in germination at 18 hours was 15% and at 24 hours was 3%. For a plasma exposure time of 2 minutes, the increase in germination at 18 hours was 18% and at 24 hours was 11%, and for a plasma exposure time of 3 minutes, the increase in germination at 18 hours was 16% and at 24 hours was 11%.

TABLE II

| Plasma Exposure | +Germination % | +Germination % |
|---|---|---|
| (Min) | (18 hours) | (24 hours) |
| 0.5 | 9 | 1 |
| 1 | 15 | 3 |
| 2 | 18 | 11 |
| 3 | 16 | 11 |

Unless otherwise indicated herein, all sub-embodiments and optional embodiments are respective sub-embodiments and optional embodiments to all embodiments described herein. While the present application has been illustrated by the description of embodiments thereof, and while the embodiments have been described in considerable detail, it is not the intention of the applicants to restrict or in any way limit the scope of the appended claims to such detail. Additional advantages and modifications will readily appear to those skilled in the art. Therefore, the application, in its broader aspects, is not limited to the specific details, the representative compositions or formulations, and illustrative examples shown and described. Accordingly, departures may be made from such details without departing from the spirit or scope of the applicant's general disclosure herein.

To the extent that the term “includes” or “including” is used in the specification or the claims, it is intended to be inclusive in a manner similar to the term “comprising” as that term is interpreted when employed as a transitional word in a claim. Furthermore, to the extent that the term “or” is employed (e.g., A or B) it is intended to mean “A or B or both.” When the applicants intend to indicate “only A or B but not both” then the term “only A or B but not both” will be employed. Thus, use of the term “or” herein is the inclusive, and not the exclusive use. Also, to the extent that the terms “in” or “into” are used in the specification or the claims, it is intended to additionally mean “on” or “onto.” Furthermore, to the extent the term “connect” is used in the specification or claims, it is intended to mean not only “directly connected to,” but also “indirectly connected to” such as connected through another component or components.

As used in the description of the invention and the appended claims, the singular forms “a,” “an,” and “the” are intended to include the plural forms as well, unless the context clearly indicates otherwise.

1. A plasma treatment device for reducing bacterial viability, enhancing stem growth, enhancing root growth or increasing germination times comprising:
a cylindrical housing that rotates about an axis
one or more medium activation nozzles;
the one or more medium activation nozzles having
a housing;
a medium inlet;
an outlet;
a high voltage electrode;
a medium passage located at least partially between the housing and the high voltage electrode and extending from the medium inlet to the medium outlet;
wherein, when the high voltage electrode is energized, a non-thermal plasma is generated in at least a portion of the medium passage; and
wherein at least a portion of the medium is activated by passing through the non-thermal plasma and creates reactive species in the activated medium.

2. The plasma treatment device of claim 1 further comprising a dielectric barrier at least partially surrounding the high voltage electrode.

3. The plasma treatment device of claim 1 further comprising a source of fluid for providing fluid to the medium inlet.

4. The plasma treatment device of claim 3 wherein the fluid comprises a mist.

5. The plasma treatment device of claim 3 wherein the fluid comprises a gas.

6. The plasma treatment device of claim 3 wherein the fluid comprises a mixture of a gas and a liquid.

7. (canceled)

8. (canceled)

9. The plasma treatment device of claim 1 comprising a plurality of medium activation nozzles located at least partially within the cylindrical housing.

10. The plasma treatment device of claim 1 further comprising at least one of a screen or at least one tumbling bar.

11. A plasma treatment device for reducing bacterial viability, enhancing stem growth, enhancing root growth or increasing germination times comprising:
a contact surface for moving crops or seeds
a plurality of medium activation nozzles;
the plurality of medium activation nozzles having
a housing;
a medium inlet;
an outlet;
a high voltage electrode; and
a medium passage located at least partially between the housing and the high voltage electrode;
wherein, when the high voltage electrode is energized, a non-thermal plasma is generated in at least a portion of the medium passage; and
wherein at least a portion of the medium is ionized to create reactive species in the medium.

12. The plasma treatment device of claim 11 wherein the contact surface is cylindrical.

13. The plasma treatment device of claim 11 wherein the contact surface is a conveyor belt.

14. (canceled)

15. (canceled)

16. (canceled)

17. (canceled)

18. (canceled)

19. (canceled)

20. A treated sprout plant treated with a plasma activated medium or with a plasma with decreased bacterial viability relative to an untreated sprout plant, the treated sprout plant comprising at least a 1-log reduction in bacterial viability relative to the untreated sprout plant.

21. (canceled)

22. The treated sprout plant of claim 20, wherein the treated sprout plant comprises at least a 3-log reduction in bacterial viability.

23. The treated sprout plant of claim 22, wherein the treated sprout plant comprises at least a 5-log reduction in bacterial viability.

24. (canceled)

25. The treated sprout plant of claim 20, wherein the treated sprout plant has decreased bacterial viability of at least one of E. coli and S. aureus.

26. The treated sprout plant of claim 25, wherein the treated sprout plant has decreased bacterial viability of both E. coli and S. aureus.

27. (canceled)

28. (canceled)

29. (canceled)

30. (canceled)

31. (canceled)

32. A method of decreasing the viability of bacteria on a sprout plant the method comprising:
exposing a sprout plant to a plasma activated medium or a plasma, thereby reducing the viability of bacteria present on the sprout plant prior to the exposure.

33. The method of claim 32, wherein the medium is selected from a gas and/or a fluid.

34. The method of claim 32, wherein the viability of the bacteria is reduced by at least 1-log following the exposure.

35. The method of claim 34, wherein the viability of the bacteria is reduced by at least 3-log following the exposure.

36. (canceled)

37. (canceled)

38. (canceled)

39. (canceled)

40. (canceled)
